(19) United States
(12) Patent Application Publication
(10) Pub. No.: US 2020/0022474 A1
(43) Pub. Date: Jan. 23, 2020

(54) MASS CASUALTY INCIDENT BAG, SYSTEM, AND METHODS OF USE

(71) Applicant: TYTEK MEDICAL INC., Cincinnati, OH (US)

(72) Inventors: Christopher C. TYLER, Cincinnati, OH (US); Richard GRIMSLEY, Cincinnati, OH (US)

(21) Appl. No.: 16/498,533

(22) PCT Filed: Mar. 27, 2018
(86) PCT No.: PCT/US18/24424

§ 371 Date: Sep. 27, 2019

Related U.S. Application Data
(60) Provisional application No. 62/478,649, filed on Mar. 30, 2017.

(51) Int. Cl. A45C 15/06; A45C 5/02; A45C 13/10; A45C 5/14; A45C 9/00; A45C 11/00; A45C 13/00; A61F 17/00

(52) U.S. Cl. CPC A45C 15/06; A45C 5/02; A45C 13/103; A45C 5/14; A45C 2013/1015; A45C 11/00; A45C 13/005; A61F 17/00; A45C 9/00

(57) ABSTRACT

A Mass Casualty Incident (MCI) Bag is provided which is a medical supply bag specifically designed to provide emergency supplies to emergency medical technicians in the event of a mass casualty event such as in a public gathering. The MCI bag is designed to allow two medical technicians to treat two or more patients each. The MCI bag is a two-sided, two bag configuration and is designed with identical sets or substantially identical sets of supplies in each bag with some specialty supplies housed in a interconnecting center portion of each bag, where they can be accessed and shared by the two technicians. In one embodiment, the MCI bag is equipped with individual flood lights for each technician to illuminate the accident scene and his patients without the need for hand held flashlights or head lamps. Also provided are a system comprising MCI carts designed to carry multiple MCI bags, and a method of responding to a Mass Casualty Incident, where the method comprises positioning prior to an incident or deploying after an incident, a plurality of MCI carts, each containing a plurality of MCI bags, around the event or incident area.

The World Bank reported in 2005 that on aggregate, the reported number of natural disasters worldwide has been rapidly increasing, from fewer than 100 in 1975 to more than 400 in 2005. Further, terrorism, as well as natural disasters, stretches the ability of emergency personnel to treat those effected by these mass casualty incidents. Thus, there is a need to provide equipment which will enable the on-site treatment of persons effected by these incidents. “Mass Casualty Incident” (MCI) can be defined as any event; planned or unplanned that results in the need to provide medical care to patients outside of traditional hospital settings. Broadly, incidents are divided into planned events (special events—like a sporting event or political event) and unplanned incidents (such as terrorism, earthquakes, natural disasters, or weather related triggering mechanisms).

### SUMMARY

In one aspect, a Mass Casualty Incident (MCI) bag is provided, comprising a support frame comprising a substantially rectangular base and a central vertical section, and two bags disposed on the substantially rectangular base, and positioned back to back against the central vertical section, of the support frame. In various embodiments, the bags of the MCI bag are substantially box-shaped, having a top, bottom, rear, front, and side panels, and the two bags are substantially the same size. Further, each bag of the two bags comprises a front portion attached to a rear portion, where the rear portion is attached to the central vertical portion of the support frame, and where the front and rear portions are attached by a coupling means. When the coupling means is uncoupled, the front portion of the bag folds down, exposing an interior of the bag and a front compartment of the bag, where each of the two bags comprises medical supplies specifically directed to a MCI and selected from bandages, tourniquets, dressings, other medical supplies, and/or combinations thereof, and are stowed in elastic mesh pocket(s) and/or elastic strap(s) located on an inside surface(s) of the bags. Each of the two bags of the MCI bag further comprise an opening in the rear panel of the bag, such that when the two bags are positioned on the support frame, the openings substantially align, and a specialized bag is disposed in an aperture created by the substantially aligned openings, and held in place by retaining means, such that the specialized bag is accessible from each of the two bags when in the open position. In one embodiment, the specialized bag contains airway supplies, and allows a technician positioned in front of each of the two bags to access and share the airway supply bag. In one embodiment, each of the two bags of the MCI bag has an internal lighting system.

In various embodiments, the support frame of the MCI bag further comprises an external lighting system comprising at least one extendable telescopic mast with a proximal end of the telescopic mast positioned at the substantially rectangular base, passing through at least a portion of the central vertical section, and a distally attached directional light, where the at least one extendable telescopic mast(s) is stored in a collapsed state between the two bags, a carrying handle attached to the top of the central vertical section, and a power supply, such as a rechargeable battery, which provides power to the external and internal lighting systems. In an alternative embodiment, the external lighting system components are contained in each of the bags of the MCI bag. In this embodiment, the power supply, which is in each of the bags of the MCI bag, powers both the external and internal lighting systems.

In another aspect, a Mass Casualty Incident (MCI) cart comprising a box with a lid and containing a plurality of Mass Casualty Incident (MCI) bags of claim 1, is provided, where the MCI cart comprises wheels or slots for forklift positioned on the bottom of the MCI cart, and/or hooks for crane lift, and where the MCI cart further comprises at least one of the following features: a) strobe lights positioned around an outside perimeter of the box; b) a window on the lid for observing an electronics module readout/indicators; c) a retaining slot for the electronics module; and c) a sensor which indicates if/when the cart is opened. In one embodiment, the electronics module comprises at least one of the following features: a) a GPS and WiFi antenna; b) a battery; c) battery charger connector; d) indicator lights, such as power, status, fault, etc.; e) a battery monitor; f) a strobe driver; and g) security transmission devices which detect and transmit opening of the MCI cart.

Also provided is a system for responding to a Mass Casualty Incident, the system comprising a plurality of the described MCI carts, each of which contains a plurality of the described MCI bags.

A method for responding to a Mass Casualty Incident is also provided, and comprises positioning prior to an incident or deploying after an incident, a plurality of described MCI carts, each containing a plurality of described MCI bags, around the event or incident area.

### BRIEF DESCRIPTION OF THE DRAWINGS

FIGS. 1A and 1B show a MCI bag according to one embodiment, in both the closed (FIG. 1A) and open (FIG. 1B) configurations.

FIG. 2 shows a support frame according to one embodiment.

### DETAILED DESCRIPTION

In one aspect, a Mass Casualty Incident (MCI) Bag is provided which is a medical supply bag specifically designed to provide emergency supplies to emergency medical technicians in the event of a mass casualty event such as in a public gathering. The MCI bag is designed to allow two medical technicians to treat two or more patients each. The MCI bag is a two-sided, two bag configuration and is designed with identical sets or substantially identical sets of supplies in each bag with some specialty supplies housed in a interconnecting center portion of each bag, where they can be accessed and shared by the two technicians. In one embodiment, the MCI bag is equipped with individual flood lights for each technician to illuminate the accident scene and his patients without the need for hand held flashlights or head lamps.

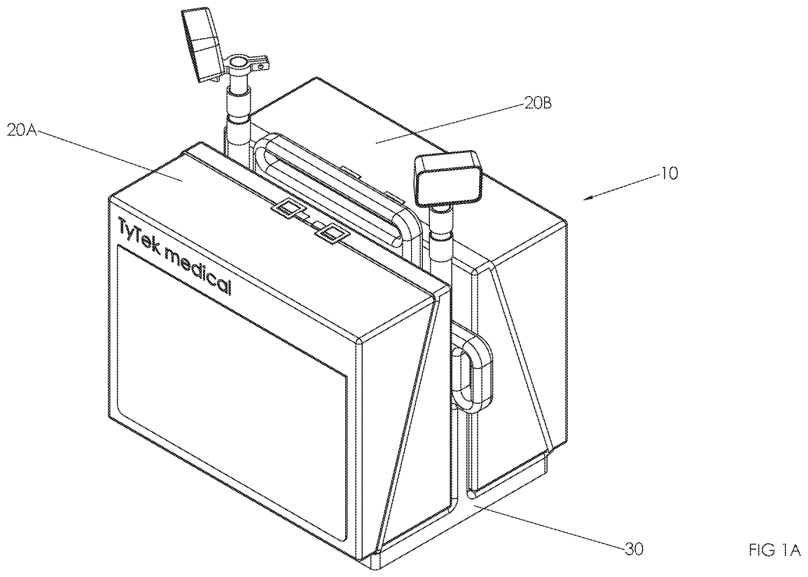

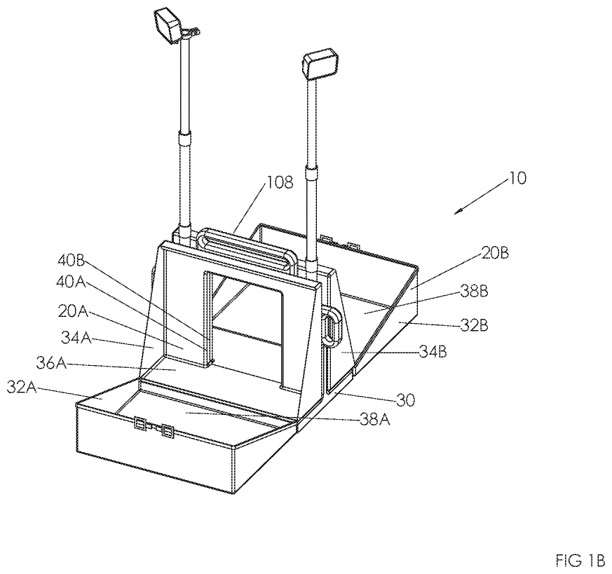

As shown in FIGS. 1A and 1B, the MCI bag (10) comprises two bags (20A, 20B) mounted back to back on opposite sides of a support frame (30). In one embodiment, the bags are constructed of a fabric material, such as nylon. In various embodiments, the bags (20A, 20B) are substantially box-shaped, and having a top, bottom, rear, front, and side panels. In various embodiments, the two bags are substantially the same size. Each bag comprises a front portion (32A, 32B) attached to a rear potion (34A, 34B), where the rear portion (34A, 34B) is disposed on a portion of the support frame. In one embodiment, the front and rear portions are attached by a zipper or other coupling means, such as Velcro. In one embodiment, the zipper or other coupling means are disposed across the top and down the side panels on each side. In one embodiment, the zipper runs down each side panel parallel to the front panel. In another embodiment, the zipper runs in a nonparallel direction on the sides. For example, on the sides, the zipper runs from a rearward position to a frontward position as it travels from top to bottom. When the zipper or other coupling means is unzipped or undone, the front of the bag folds down exposing the interior of the bag (36A, 36B) and front compartment (38A, 38B).

Contents of the bag, which are specifically directed to use in a MCI, and including bandages, tourniquets, dressings and various other medical supplies, are stowed in numerous elastic mesh pockets or elastic straps located on the inside surfaces of the bags. In various embodiments, the MCI-specific medical supplies comprise bandages, burn-related medical supplies, airway-related control instruments and supplies, etc. In various embodiments, the contents comprise a plurality of the following: cricothyroidotomy kit, i-gel lube, i-gel supraglottic (large, medium, and small), nasal pharyngeal airway designed to be inserted into the nasal passageway to secure an open airway (NPA; large, medium, and small), oropharyngeal airway which is a tubular device used to maintain or open a patient's airway, by preventing the tongue from covering the epiglottis which would prevent the person from breathing (OPA; large, medium, and small), a manual resuscitator which is a device used to provide positive pressure ventilation to patients who are not breathing or not breathing adequately, such as a pocket BVM, a tension pneumothorax access needle (decompression needle) used to access the pleural cavity and release the buildup of air resulting from an open chest wound or lung laceration, such as TVAC, chest seal—Halo dressing, Coban—Coflex (Vet Wrap) 3″ and 4″, packing gauze, a structural aluminum malleable splint which is a compact device used for immobilizing bone and soft tissue injuries in a trauma setting (SAM), tourniquet, triangle bandages, watergel, biohazard bag, duct tape, flash light, gloves, such as nitrile, heating blanket, patient movement device, such as a tarp or Megamover, which is a rugged device similar to a stretcher but used to drag a patient to a safe location, Saran Wrap, shears, surveyors tape, and tarp. The supplies are arranged on the back and side walls of the interior of the bag and in the front compartment. In one embodiment, there is a pocket located on the outside of the front of each bag that is used to store larger flat supplies such as heat blankets, transport devices etc.

In one embodiment, each of the bags (20A, 20B) have an opening (40A, 40B) in the rear panel of the bag, such that the openings (40A, 40B) substantially align. In one embodiment, a specialized bag is disposed in the aperture created by the openings, and held in place by retaining means, such as a Velco strap, such that the specialized bag is accessible from both bags (20A, 20B) when in the open position. In one embodiment, the specialized bag contains airway supplies, and allows each technician to access and share the airway supply bag.

In various embodiments, the bags (20A, 20B) are constructed of a fabric, such as nylon, and may additional be lined to provide protection for the supplies in the bag. In one embodiment, the interior of the bags (20A, 20B) are light reflective fabric. The light reflective fabric helps to provide improved illumination to the interior of the bag and its supplies.

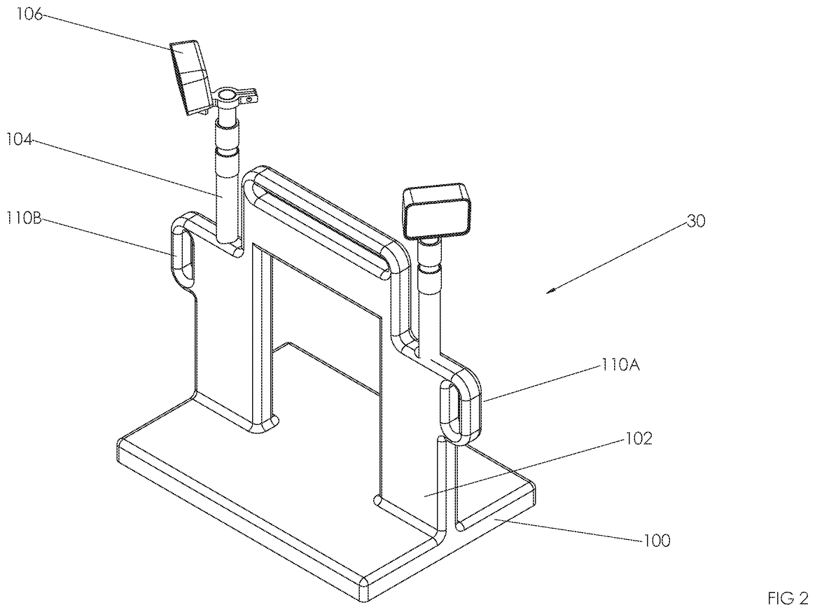

The support frame (30), as shown in one embodiment in FIG. 2, comprises a rectangular base (100) and a center vertical section (102) extending up from the center of the base. The two bags (20A, 20B) sit on opposite sides of the base of the frame. The bags (20A, 20B) are positioned back to back against the vertical section of the frame. The rear panel of the bags (20A, 20B) is secured to the vertical section of the frame (102). The bags (20A, 20B) may be secured to the vertical section of the frame (102) using screws or other fastening means such as cord lacing, ties, rivets etc. The support frame (30) is constructed of a rigid planar or tubular material, such as rectangular tubing or circular tubing. In one embodiment, the support frame is fabricated from carbon fiber. In other embodiments, the support frame (30) is constructed of extruded aluminum sections, welded aluminum tubing, formed sheet metal, and/or molded plastic/fiberglass/carbon fiber. In one embodiment, all or a portion of the support frame (30) is covered with a plastic coating. Attached to the rectangular base (100) is the central vertical section (102) that supports the back of the bags (20A, 20B) and an extendable telescopic mast (104) which have mounted work light(s) (106). This allows each side of the MCI bag (10) to have an independent lighting system comprising a work light (106), which is directional, mounted on an extendable mast (104). In one embodiment, each of the bags (20A, 20B) also has interior lighting to illuminate the interior of the bag. A carrying handle (108, as shown in FIG. 1B) is also attached to the top of the central vertical section (102). This handle allows a single technician to transport the MCI bag to the MCI scene. In an additional embodiment, carrying handles (110A, 110B) are also positioned on the sides of the MCI bag to allow, for instance, transporting of the MCI bag by two technicians.

The exterior lighting system comprising the work lights (106) mounted on the extendable telescopic masts (104) allow a technician to illuminate the patient(s) without the need for head lamps or flash lights. In one embodiment, two work lights (106) are mounted on extendable masts (104) that extend out of opposite sides of the rectangular base (100). The extendable masts (104) are stored in a collapsed state between the two bags and in one embodiment, can be extended up to about 5 feet. The work light height, direction and tilt are fully adjustable. The extendable mast mounted work lights are independent of one another and have individual ON/OFF controls. In an additional embodiment, the external lighting system components are located in each of the bags of the MCI bag. In one embodiment, the extendable mast is positioned substantially horizontally inside the bag, with the proximal end attached to a hinge, such that once the bag is opened, the extendable mast can be moved from the substantially horizontal position to a vertical position, and then extended to a desired height. In this alternative embodiment, the height of the bag may be decreased, thus allowing for easier storage, as well as decreasing the weight/complexity of the support frame. Further, in this embodiment, the support frame can be constructed of panels or substantially flat materials.

In one embodiment, an interior lighting system is provided inside the bag to illuminate the interior of the bag. In one embodiment, the interior lighting system comprises a ‘rope light’, such as a string of LED indicators. The interior light system is mounted to illuminate the interior of the bag, such as on the inside of the bag at the top. When turned on, the interior lighting system will illuminate the back of the bag and the inside of the front of the bag. In one embodiment, the interior of the bag is constructed of reflective material to reflect light inside the bag. A light shield may be installed in front of the internal lighting system so the light is blocked from being directly in the eyes of the EMT. In one embodiment, the interior lighting system is controlled by the same switch that controls the external lighting system and its mast mounted work light. In an additional embodiment, the internal and external lighting systems are activated by the opening of the bag and/or lowering the front panel of the opened bag.

In various embodiments, the MCI bag also includes a power supply. In one embodiment,

A 12 volt rechargeable battery is contained within the rectangular base (100) of the support frame (30), and provides power, such as to the external and internal lighting systems. In an alternative embodiment, the power supply is located in each of the bags of the MCI bag. In one embodiment, the rechargeable battery is designed to provide about 1 hour or more of continuous use of both internal and external lighting systems. In one embodiment, the rechargeable battery is charged by an external 12 volt charger powered by 115/230 volt mains voltage.

In one embodiment, the MCI bag comprises two fabric bags and a rigid support frame. In various embodiments, the fabric bags have at least one of the following features: a) front of each bag has a zippered opening, the opening being the entire side of the bag; b) the fabric bags are secured to the rigid frame, such as by Velcro loops or snaps; an opening in the back of each bag, allowing access from one bag to the other, and in one embodiment, Velcro on the opening of each bag to maintain relative position of the bags; and d) optional plastic supports inside bag sides to resist the bag collapsing. In an additional embodiment, the bags of the MCI bag also contain fold-out panel(s) constructed of a relatively rigid material, where one side of the fold-out panel is attached/hinged to the bag, such that when the bag is in the open position, the fold-out panel can be rotated to an open position. In this embodiment, the interior surface area of the bag is increased, thus allowing for additional pockets for supplies, etc.

The rigid support frame is disposed between and underneath the two fabric bags, such that the fabric bags are positioned back to back on the frame. In various embodiment, the rigid support frame has at least one of the following features: a) a carry handle positioned on the top of the rigid base and/or sides of the bag; b) one or two LED light panels connected to the rigid base via a telescopic mast, and in a retracted position, stowable in the rigid frame, where in some embodiments, the telescopic mast has a lock to maintain the light at a desired height, where both masts can be positioned independently; and c) a battery, and optionally a charger, in base of the rigid support frame. In one embodiment, the telescopic mast is extended automatically when the bag is opened, using, for example, a motor-actuated or spring-loaded mechanism.

In a further aspect, the described MCI bags are stored in a MCI cart, which is designed to carry multiple MCI bags, as well as additional supplies for a MCI. In various embodiments, the cart contains, in addition to the MCI bags, at least one box of gloves, such as nitrile (small, medium, and large), marker board with pens, shroud, such as a body bag, and a stretcher. In various embodiments, the MCI cart is a box with a lid, and having wheels or slots for forklift positioned on the bottom of the MCI cart, and/or hooks/slots for crane lift. In various embodiments, the MCI cart has at least one of the following features: a) strobe lights positioned around the outside perimeter of the box; b) a window on the lid for observing an electronics module readout/indicators; c) a retaining slot for the electronics module; and c) a sensor which indicates if/when the Cart is opened. The electronics module, which in one embodiment is slidable into the MCI cart, has at least one of the following features: a) a GPS and WiFi antenna; b) a battery; c) battery charger connector; d) indicator lights, such as power, status, fault, etc.; e) a battery monitor; f) a strobe driver; and g) security transmission devices which detect and transmit opening of the MCI cart.

In various embodiments, the security of the carts is monitored to ensure the carts are not tampered with or moved, as well as indicating whether the carts have been opened. The GPS position data can be constantly monitored by a master controller system to ensure the cart has not been moved. Further, each cart can have a unique ID number so it can be tracked and its location noted on an electronic map.

In another aspect, a system is provided which comprises a plurality of the described MCI carts positioned around an event area, such as a sporting event venue, with each MCI cart containing a plurality of the described MCI bags. In various embodiments, the system further comprises a master controller which is in communication with the MCI carts, and provides positioning information of the MCI carts, as well as when/if an MCI cart has been accessed/opened. In a further embodiment, the system may comprise different types of MCI carts, such that some carts contain different equipment/supplies and/or MCI bags with different equipment/supplies appropriate for the location of the MCI cart. For example, deployment of the described system at a racetrack may include a specialized MCI cart and/or MCI bags positioned at the “pits” of a racetrack.

In another aspect, a method of responding to a Mass Casualty Incident is provided, where the method comprises positioning prior to an incident or deploying after an incident, a plurality of MCI carts, each containing a plurality of MCI bags, around the event or incident area. The method may further comprise communicating, from a master controller, with the positioned plurality of MCI carts.

What is claimed is:

1. A Mass Casualty Incident (MCI) bag comprising
a support frame comprising a substantially rectangular base and a central vertical section, and
two bags disposed on the substantially rectangular base, and positioned back to back against the central vertical section, of the support frame.

2. The MCI bag of claim 1 wherein the bags are constructed of a fabric material.

3. The MCI bag of claim 2 wherein the bags are constructed of nylon.

4. The MCI bag of claim 1 wherein the bags are constructed of a substantially rigid material.

5. The MCI bag of claim 4 wherein the bags are constructed of molded plastic, fiberglass, carbon-fiber or other moldable material.

6. The MCI bag of claim 1 wherein the bags are substantially box-shaped, and having a top, bottom, rear, front, and side panels.

7. The MCI bag of claim 1 wherein the two bags are substantially the same size.

8. The MCI bag of claim 1 wherein each bag of the two bags comprises a front portion attached to a rear portion, where the rear portion is attached to the central vertical portion of the support frame.

9. The MCI bag of claim 8 wherein the front and rear portions are attached by a coupling means.

10. The MCI bag of claim 9 wherein the coupling means is a zipper or Velcro.

11. The MCI bag of claim 9 wherein the coupling means are disposed across the top and down the side panels on each side of each bag.

12. The MCI bag of claim 11 wherein the coupling means on each side panel is parallel or nonparallel to the front panel.

13. The MCI bag of claim 9 wherein when the coupling means is uncoupled, the front portion of the bag folds down, exposing an interior of the bag and a front compartment of the bag.

14. The MCI bag of claim 1 wherein each of the two bags comprises medical supplies specific for an MCI and selected from bandages, tourniquets, dressings, other medical supplies, and/or combinations thereof.

15. The MCI bag of claim 14 wherein the medical supplies are stowed in elastic mesh pocket(s) and/or elastic strap(s) located on an inside surface(s) of the bags.

16. The MCI bag of claim 15 wherein the medical supplies are arranged on a back and side walls of the interior of the bag and in the front compartment.

17. The MCI bag of claim 14, wherein the bag further comprises fold-out panels, and the medical supplies are also arranged on the surface(s) of the fold-out panel.

18. The MCI bag of claim 6 further comprising a pocket located on the outside of the front of each bag that is used to store large flat supplies.

19. The MCI bag of claim 18 wherein the large flat supplies are heat blankets and/or transport devices.

20. The MCI bag of claim 6 wherein each of the two bags further comprises an opening in the rear panel of the bag, such that when the two bags are positioned on the support frame, the openings substantially align.

21. The MCI bag of claim 20 wherein a specialized bag is disposed in an aperture created by the substantially aligned openings, and held in place by retaining means, such that the specialized bag is accessible from each of the two bags when in the open position.

22. The MCI bag of claim 21 wherein the specialized bag contains airway supplies, and allows a technician positioned in front of each of the two bags to access and share the airway supply bag.

23. The MCI bag of claim 6 wherein each of the two bags further comprises an additional lining material.

24. The MCI bag of claim 13 wherein the interior of each of the two bags is lined with a light reflective fabric, to provide improved illumination to the interior of the bag and its supplies.

25. The MCI bag of claim 1 wherein a rear panel of each of the two bags is secured to the central vertical section of the support frame.

26. The MCI bag of claim 25 wherein each of the two bags is secured to the central vertical section of the support frame by screws, cord lacing, ties, and/or rivets etc.

27. The MCI bag of claim 1 wherein the support frame is constructed of rectangular or circular tubing or panels.

28. The MCI bag of claim 27 wherein the tubing or panels is fabricated from carbon fiber, extruded aluminum, welded aluminum, formed sheet metal, molded plastic/fiberglass/carbon fiber, and/or combinations thereof.

29. The MCI bag of claim 28 wherein all or a portion of the support frame is covered with a plastic coating.

30. The MCI bag of claim 1 wherein the support frame further comprises an external lighting system comprising at least one extendable telescopic mast with a proximal end of the telescopic mast positioned at the substantially rectangular base, passing through at least a portion of the central vertical section, and a distally attached directional light.

31. The MCI bag of claim 30 wherein the external lighting system comprises two extendable telescopic masts, each with a distally attached directional light, with the proximal end of each extendable telescopic mast positioned on opposite sides of the rectangular base.

32. The MCI bag of claim 30 wherein the at least one extendable telescopic masts is stored in a collapsed state between the two bags.

33. The MCI bag of claim 30 wherein the at least one extendable telescopic mast is extendable up to about 5 feet.

34. The MCI bag of claim 30 wherein the at least one extendable telescopic mast provides work light height, direction, and tilt which are fully adjustable.

35. The MCI bag of claim 30 wherein the directional light(s) are activated by an ON/OFF switch located in the substantially rectangular base of the support frame.

36. The MCI bag of claim 30 wherein the directional light(s) are activated by opening the bag and/or moving the front panel of the bag into the open position.

37. The MCI bag of claim 30 wherein the at least one extendable telescopic mast is extended automatically by opening the bag and/or moving the front panel of the bag into the open position.

38. The MCI bag of claim 1 wherein each of the bags comprises an external lighting system comprising at least one extendable telescopic mast with a proximal end of the telescopic mast secured to the bottom of the bag and a distally attached directional light.

39. The MCI bag of claim 38 wherein the proximal end of the telescopic mast is attached to the bottom of the bag by a hinge, where the telescopic mast is stored in a substantially horizontal position in the bag, and is movable to a vertical position.

40. The MCI bag of claim 1 wherein each of the two bags further comprise an interior lighting system.

41. The MCI bag of claim 40 wherein the interior lighting system comprises a string of LED lights.

42. The MCI bag of claim 40 wherein the interior lighting system is mounted on the inside of the bag at the top panel.

43. The MCI bag of claim 42 wherein a light shield is positioned in front of the interior lighting system to reduce the amount of light being directly in the eyes of a user.

44. The MCI bag of claim 40 wherein the internal lighting system is activated by an ON/OFF switch located in the substantially rectangular base of the support frame.

45. The MCI bag of claim 40 wherein the internal lighting system is activated by opening the bag and/or moving the front panel of the bag into the open position.

46. The MCI bag of claim 44 wherein the ON/OFF switch for the internal lighting system is the same as the ON/OFF switch for the external lighting system.

47. The MCI bag of claim 1 wherein the support frame further comprises a carrying handle attached to the top of the central vertical section.

48. The MCI bag of claim 1 wherein the MCI bag further comprises carrying handles positioned on the sides of the MCI bag.

49. The MCI bag of claim 1 further comprising a power supply.

50. The MCI bag of claim 49 wherein the power supply is a rechargeable battery and is contained within the substantially rectangular base of the support frame or is contained in each of the bags of the MCI bag.

51. The MCI bag of claim 50 wherein the power supply supplies electricity to the external lighting system and the internal lighting system.

52. A Mass Casualty Incident (MCI) cart comprising a box with a lid and a plurality of Mass Casualty Incident (MCI) bags of claim 1.

53. The MCI cart of claim 52 further comprising wheels or slots for forklift positioned on the bottom of the MCI cart and/or hooks/slots for crane transport.

54. The MCI cart of claim 52 wherein the MCI cart further comprises at least one of the following features: a) strobe lights positioned around an outside perimeter of the box; b) a window on the lid for observing an electronics module readout/indicators; c) a retaining slot for the electronics module; d) a sensor which indicates if/when the cart is opened, and e) supplies specific for an MCI.

55. The MCI cart of claim 54 wherein the electronics module comprises at least one of the following features: a) a GPS and WiFi antenna; b) a battery; c) battery charger connector; d) indicator lights, such as power, status, fault, etc.; e) a battery monitor; f) a strobe driver; and g) security transmission devices which detect and transmit opening of the MCI cart.

56. The MCI cart of claim 54 wherein the supplies specific for an MCI comprise a stretcher.

57. A system for responding to a Mass Casualty Incident, the system comprising a plurality of the MCI carts of claim 52.

58. A method for responding to a Mass Casualty Incident, the method comprising positioning prior to an incident or deploying after an incident, a plurality of MCI carts of claim 52, each containing a plurality of MCI bags of claim 1, around the event or incident area.

59. The method of claim 58 further comprising communicating, from a master controller, with the positioned plurality of MCI carts.

60. The method of claim 59, further comprising continuous monitoring of cart security status, including whether the cart has been opened and the position of the cart.
